(19) United States
(12) Patent Application Publication
(10) Pub. No.: US 2003/0225316 A1
(43) Pub. Date: Dec. 4, 2003

(54) Method and system for measuring the success of treatment of a medical therapy

(76) Inventors: Klaus Abraham-Fuchs, Erlangen (DE); Uwe Eisermann, Erlangen (DE); Achim Hein, Nuernberg (DE); Niels Richter, Thurnau (DE); Robert Setz, Rednitzhembach (DE)

Correspondence Address: HARNESS, DICKEY & PIERCE, P.L.C., P.O.BOX 8910, RESTON, VA, 20195, US

(21) Appl. No.: 10/410,162

(22) Filed: Apr. 10, 2003

(30) Foreign Application Priority Data
Apr. 10, 2002 (EP) 02008042.0

(51) Int. Cl. A61B 5/00

(52) U.S. Cl. 600/300

(57) ABSTRACT

A method and a system are for measuring the success of treatment of a medical therapy. In the method, at least one treatment objective is defined at a first data processing station and a calculation rule is provided for the success of treatment of the therapy, the calculation rule indicating the success of treatment as a function of recordable numerical measures. Furthermore, a second data processing station is provided for the automated recording and transmission of the numerical measures describing the success of treatment to the first data processing station. At the start of the therapy, the initial state of the patient with respect to the treatment objective is recorded using first numerical measures. During and/or at the end of the treatment, automatic second numerical measures describing the success of treatment are recorded by way of the second data processing station. The success of treatment is automatically calculated with reference to these numerical measures and represented at the first data processing station or at a further data processing station. The method permits reliable and automated measurement of the success of treatment of a medical therapy, in particular in the field of telemedical forms of treatment, and can be advantageously integrated into the normal course of therapy.

The present application hereby claims priority under 35 U.S.C. §119 on European patent application number EP 02008042.0 filed Apr. 10, 2002, the entire contents of which are hereby incorporated herein by reference.

### FIELD OF THE INVENTION

The present invention generally relates to a method and to a system for measuring the success of treatment of a medical therapy such as is carried out in particular within the scope of rehabilitation or in the context of Disease Management Services for patients with chronic illnesses. A particular field of application is telemedical forms of treatment in which the patient carries out training units, of which the therapy is composed, in his domestic environment and is connected to the attending physician or therapist only via a data link.

### BACKGROUND OF THE INVENTION

Recent developments in health services, in particular evidence-based medicine, medical guidelines and the introduction of the DRG (Disease Related Groups) system, are increasingly resulting in a wish to find an objective way of measuring the success of a medical treatment. Such a measurement of the success of treatment, also referred to below as Outcome, can provide a decision aid for further procedures during the treatment. A standardized measurement of the success of treatment can also be used for the comparison of the quality of different medical service providers.

Hitherto, the success of treatment of a medical therapy has usually been measured only within the scope of clinical studies, but not within the everyday routine of the provision of medical care. Furthermore, in such studies, there is generally a restriction to partial aspects of the treatment of manageable scope, for example to a comparison between two courses of medication or to a comparison between two surgical procedures.

WO 96/30848 describes a system for automatically producing a report on the state of health and on recommended therapeutic measures for a patient with cardiac problems. However, the publication is not concerned with the measurement of the success of treatment of a therapy.

WO 00/75853 discloses a digital illness management system which automatically issues a recommendation as to whether or not immediate treatment would provide advantages for the patient, on the basis of input patient data, including data on the instantaneous state of health of the patient and cost and administration information. This is achieved by accessing a database which contains individual patient data on a multiplicity of patients. This publication is therefore not concerned with a method or system for measuring the success of treatment of a therapy either.

U.S. Pat. No. 5,582,186 relates to a method for automatically analyzing the spinal column of a patient by recording measured data relating to a rotational movement of the spinal column of the patient and to a healthy person for comparison, the data items which are recorded on both sides being compared with one another graphically in order to detect possible anomalies of the patient. This publication is therefore not concerned with the measurement of the success of treatment of a therapy either.

U.S. Pat. No. 5,524,645 describes a method for monitoring the therapeutic progress of a medical treatment and the efficiency of the rehabilitation process. Here, parameters which are linked to the region of the body on which therapy is to be performed are defined, the parameters being capable of quantification by way of recordable numerical measures. Using a calculation rule, a composite value, which constitutes a measure of the state of the region of the body on which therapy is to be performed is calculated from the numerical measures. The numerical measures are recorded at the start and repeatedly during the medical treatment, and the respectively corresponding composite value is calculated. The course of the therapy and the success of the therapy as well as the efficiency of the therapy can be displayed by graphically representing the composite values in comparison with an ideal value. In one refinement of the disclosed method, the costs of the therapy are also included in the measurement of the success of treatment.

U.S. Pat. No. 5,435,324 is concerned with a method and a device for measuring the progress or Outcome of a psychotherapy in which numerical measures relating to the state of the patient are measured and combined to form an index which is a measure of the success/progress of the therapy. The numerical measures are repeatedly measured during the therapy and the corresponding indices calculated therefrom. The respectively stored index values are used to assess the progress of the treatment with respect to the initial state or a benchmark value which is obtained from comparison data on a multiplicity of patients in a database. Furthermore, a device is provided with which the effectiveness of the therapy is determined.

However, despite already existing solutions, there continues to be a need for a method for consistently and comparably measuring the success of treatment of a medical therapy in a way which is automated using computer support and which can be embedded in the customary clinical workflow or the disease management service as there is increasing use of new forms of telemedical treatment, to which such epithets as Telemedicine, Home care and Integrated Care are attached. Telemedicine forms of treatment are particularly suitable both for aftercare after rehabilitation measures and in the case of chronic illnesses such as, for example, diabetes, asthma or cardiac problems. They are generally characterized by the cooperative involvement of a plurality of medical service providers in a treatment process, by their complexity and by the long duration of the treatment process.

The treatment here may extend over a very long time period of months to years. In the treatment it is frequently necessary to take into account multi-morbidities, i.e. the simultaneous presence of a number of illnesses, so that the treatment is made up of a large number of component processes or measures which are only apparently independent. Although it is organizationally easier to measure the success of treatment by separately measuring the Outcome of the individual, apparently separate component processes, this frequently tends against the objective of the measurement of the success of treatment of the overall therapy, as in many cases the sum of the separately measured successes of the individual measures does not constitute a reliable measure of the success of the overall therapy. Therapy is to be understood here as the sum of the individual therapeutic measures of which the treatment is composed.

### SUMMARY OF THE INVENTION

An object of an embodiment of the present invention is to specify a method and a system for measuring the success of treatment of a medical therapy which can be integrated into the normal treatment process and which make possible a comparable and reliable measurement of the success of treatment of the selected therapy, in particular in a spatially distributed telemedical rehabilitation or treatment process.

In the present embodiments of methods for measuring the success of treatment of a medical therapy, at least one treatment objective of the therapy is firstly defined. This is preferably an objective which is superordinate to the sum of all the therapeutic measures of the therapy, for example the improved or maintained quality of life of the patient, the independence of lifestyle, a reduced degree of incapacity for work, etc. A calculation rule for the success of treatment of the therapy with respect to the treatment objective is then provided, said calculation rule indicating the success of treatment as a function of recordable numerical measures.

The numerical measures here preferably do not constitute absolute physical variables but rather relative variables, such as, for example, the percentage of attainment of the objective, the fraction of the attainment of the objective or a standardized score. They may constitute individual measured values or be derived from one or more measured values. It goes without saying that it must be possible to reliably record or measure these numerical measures—or the variables from which they are calculated.

Finally, one or more first numerical measures describing the initial state of the patient to be subjected to the therapy, with respect to the treatment objective, are recorded, preferably at the start of the therapy. The first numerical measure may constitute, for example, the measured value of the degree of an existing deficit at the start of the treatment in relative measurement units with respect to a reference variable or with respect to the size of the deficit when the treatment objective is attained. The defined treatment objective, the selected calculation rule and the first numerical measures are finally stored. This can be carried out in a database which is respectively provided for this purpose, or else in an electronic patient file. The definition of the treatment objective and the provision of the calculation rule are carried out at a first data processing station at which the appropriate means are made available to the therapist or physician.

Furthermore, a second data processing station is made available to the patient, and is preferably configured in such a way that it can record the numerical measures describing the success of treatment within the course of the therapy in a computer-supported and largely automated fashion. In order to calculate the success of treatment at any desired time within the course of the treatment or at the end of the treatment, the second data processing station records second numerical measures describing the respective deficit of the patient at this evaluation time and transmits them to the first data processing station via a network. The previously stored data is called at this first data processing station or at the second data processing station.

The automatic calculation and representation of the success of treatment is carried out using the calculation rule on the basis of this data, i.e. at least the treatment objective, the calculation rule and the first and second numerical measures. The calculation can be carried out at the first data station as well as already at the second data station from which the calculated success of treatment is then transmitted to the first data processing station in addition to, or instead of, the second numerical measures.

Of course, with the present methods it is also possible to predefine a plurality of treatment objectives which can relate to individual therapeutic measures or groups of therapeutic measures. In this case, it may possibly be necessary to make available a different calculation rule for each treatment objective, and to record different first and second numerical measures. In the case of the present method, the calculation rule may be dependent not only on a category of numerical measures of the medical result E of the treatment, expressed by the first and second numerical measures above, but also on further categories. Such categories include, for example, numerical measures of the costs K of the treatment, a numerical measure of the duration of the treatment T and a numerical measure of the compliance C of the patient. The calculation rule can thus equate to a general function of the mathematical form f (E, K, C, T). The numerical measures may, for example, also be table-like scores in which the treatment result is achieved by summing points which represent the numerical measures of the categories E, K, T and C. The points can be assigned to the measured values using what are referred to as look-up tables, for example.

In the text which follows, the present methods and preferred embodiments of these methods are clarified once more with reference to a specific calculation rule. However, it goes without saying that, in order to calculate the success of treatment from the respective numerical measures, it is also possible to use other calculation rules which indicate the success of treatment as a function of the respective numerical measures.

An important precondition for the measurement of the success of treatment is the clear and unambiguous description of a treatment objective, in which case it must be possible to measure the degree to which this treatment objective is attained. In this context, it may be advantageous to specify a plurality of partial objectives which are to be attained in various stages of the treatment process. The selection of relative numerical measures in the present methods ensures that the attainment of the individual partial objectives can be compared.

Important treatment objectives of a therapy which constitute objectives which are superordinate to the sum of all the therapeutic measures are not listed in conclusion by way of example in the text which follows. A frequent objective of a rehabilitative therapy or therapeutic measure is to reduce or eliminate a deficit in the physical and psychological capabilities due to an illness or an accident. These deficits are generally the result of weakening or the complete failure of a region of the brain, a muscle or an organ. A deficit in a capability or performance can be measured as a percentage of the loss of 100% capability of an average healthy normal person. The measurable capability may relate, for example, to the mobility of a joint in degrees, the strength of a muscle in kilograms of load-bearing capacity or the size of the field of vision in degrees in the case of visual impairments.

A further treatment objective may be to improve capacities which are important in particular for the independent care of the patient. A capacity is understood in the context of a medical rehabilitation measure as a complex action which can, however, be divided up into independent actions which are separate from other actions. A capacity requires the interaction of a plurality of capabilities. A still further objective of a rehabilitative measure may be quite generally the improvement of the quality of life of the patient. There are measuring instruments for measuring the quality of life in the form of standardized questionnaires which are filled in by the patient.

Another clearly defined objective of a treatment may also be to improve the state of the patient with respect to the need for care so that the patient can be classified in a better care level when the treatment objective is attained. The assignment to a care level is generally determined by the assessment of a plurality of capacity deficits and capability deficits. For the patient, an improved care level means he can take more control of his life, and for the health system it means lower care costs.

Yet another treatment objective may be to reduce the degree of an existing incapacity for work, which may be expressed, for example, as a numerical measure as a percentage of the incapacity for work, in order to permit the greatest possible degree of reintegration into working life. A still further treatment objective may be to improve or stabilize the malfunctioning of an organ, which can be quantified by measuring the degree to which a physiological parameter deviates from a healthy normal range, for example by measuring the blood pressure in the case of a cardiovascular illness, by measuring the heart rate at a defined load when there is a cardiac problem or by means of measurements of the tidal volume of the breath in the case of asthma.

After the treatment objective of the therapy has been defined, a database with the treatment objective or the partial objectives of the treatment, at least a measured value of a degree of at least one existing deficit D at the start of the treatment in relative measurement units and the selected calculation rule for the success of treatment is created for the patient. It is also optionally possible to store an anticipated target value of the deficit as a prognosis after the treatment has been terminated. At least at the end of the treatment, a further measured value of the deficit D is recorded in relative measurement units and used to calculate the success of treatment. It is possible to use here, for example, one of the following formulae for the quality-oriented Outcome (Q) as calculation rule:

Outcome (Q)=Result=(1−Deficit), therefore:

Outcome (Q)=1−D (End), or

Outcome (Q)=D (Start)−D (End)

In one advantageous embodiment of the present methods, the measured value of the deficit D is recorded in relative measurement units not only at the start and at the end of the treatment but also at any desired predefinable time intervals or at any desired predefinable times Ti within the course of the treatment. In this way, it is possible to represent the change at the predefined times during the treatment in comparison with the initial state. From these intermediate values of the Outcome it is also possible to generate a profile curve which provides the therapist or physician with information on the chronological profile of the success of treatment.

The recording or measurement of the respective numerical measures can be carried out using different measurement techniques or methods/devices which depend on the type of deficit to be described with the numerical measure. Thus, the measured values of the numerical measures can be recorded, for example, by an appropriate questionnaire being filled in by the patient or by a training exercise being measured using an appropriate measured value sensor on the training device. These devices are connected to the second data processing station which is installed at the location where the training or the therapeutic measures are carried out, in particular in the domestic environment of the patient, and is at least temporarily connected to the first data processing station of the therapist or physician via a network. The second data processing station is installed as a computer workstation and is configured or equipped with means in such a way that it automatically carries out, at the predefinable times, the measurements necessary for the recording of the numerical measures, or causes said measurements to be carried out, and transmits them to the first data processing station. In the opposite direction, it is also possible to bring about the recording of the necessary measured values at the second data processing station by means of the first data processing station. For specific forms of treatment, the second data processing station may, for example, also be implemented by means of a palmtop if it is suitable for recording the respective numerical measures.

Given the increasing cost pressures in health services, it is increasingly no longer sufficient for a medical service provider to consider the attainment of the medical objective alone. Instead, the attainment of the medical result must be seen in relation to the resources employed for this purpose, i.e. the costs incurred. In one of the proposed methods, a calculation rule for a cost-oriented Outcome (E, K) is therefore provided in which the treatment result attained is placed in relation to the costs incurred by it. A corresponding calculation rule may be configured, for example, in the following way:

Outcome (E, K)=Outcome (Q): Costs=E:K,

the result E being measured in the most general form as a percentage improvement in a deficit which existed at the start of the treatment, and the costs K covering all the resources used to attain the objective. In this preferred development of the present method, not only the numerical measures for the evaluation of the deficit but also numerical measures for the costs incurred within the scope of the therapy are thus taken into account. By specifying the cost-oriented Outcome (E, K) in relative units E/K, i.e. per currency unit, instead of in absolute units, this cost-oriented success of treatment can also easily be compared over a plurality of partial objectives or a plurality of health institutions or service providers.

In one developed embodiment of the method, the costs are divided into cost elements which cannot be influenced and into cost elements which are highly variable and can be influenced by measures of the service provider or by types of behavior of the patient. Cost elements which cannot be influenced are, for example, fixed costs which occur to an equal degree during each treatment, as well as overhead costs which occur, for example, in proportion to the duration of treatment, but are always approximately of the same level. Such cost elements which cannot be influenced do not need to be taken into account, or only need to be taken into account as an approximate, fixed estimated value K (fixed), while the cost elements K (var) which can be influenced are recorded specifically. The cost-oriented success of treatment Outcome (E, K) can be specified here either only as a function of the cost elements which can be influenced Outcome (E, K)=Result: K (var) or as a function of both cost elements Outcome (E, K)=Result: (K (fixed)+K (var)). This procedure minimizes the expenditure on acquiring the necessary numerical measures without the informativeness of the resulting success of treatment being compromised. In reality, acquiring all the direct and indirect costs for a rehabilitation measure would be much too costly and is also generally not necessary for an informative result as the fixed costs generally cannot be influenced by the service provider, or can hardly be influenced.

In order to calculate the cost-oriented success of treatment, actual costs are not recorded as they are rarely recorded directly in the typical workflow of a clinical process, and are thus also not available without considerable additional expenditure. Instead of the actual costs, key variables for the calculation of the costs are rather recorded in the present method, said key variables being, on the one hand, a component of the treatment workflow but, on the other hand, being highly proportional to relevant cost elements, in particular to the dominant, highly variable costs which can be influenced. Examples of such key variables are, for example, the number or the duration of personal sessions with the patient which are directly related to the costs incurred thereby. Further examples are given in one of the exemplary embodiments at the end of the present description. These key numbers which can easily be recorded may, depending on the anticipated informativeness of the cost-oriented success of treatment for the relevant variable cost elements, either be included directly in the calculation of the Outcome or be provided with weighting factors W(i) which converts the respective key factors S(i) into currency units. K (var)=W(i)×S(i). This can of course be carried out both for fixed and variable cost elements. As a result, it is generally true that:

Outcome (E, K)=E:K

Outcome (E, K)=Result: (K (fixed)+K (var)), where:

$$K = {\sum }_{j = 1}^{n} ( Wfixed ( j ) \times Sfixed ( j ) ) + {\sum }_{i = 1}^{n} ( Wfixed ( i ) \times Sfixed ( i ) ) .$$

In order to simplify the handling, the cost elements which are in any case fixed are preferably omitted from the calculation or replaced by an approximate estimated value, and only a few relevant key numbers S(i) which can easily be measured in an automated fashion are taken into account with associated weighting factors W(i) in the calculation. Basically, in order to create a calculation rule which is suitable for a rehabilitation process, for calculating the Outcome (E, K), the fixed and the variable cost elements of the respective rehabilitation process must firstly be identified here and their order of magnitude estimated. If cost elements are significantly lower than the overall costs, they can be omitted for the sake of simplification, depending on the level of accuracy required.

In one embodiment of the present methods, the time required overall for the therapy in order to attain the therapy result is also included in the calculation. Given the same level of quality of the result and identical costs in a shorter time, a better Outcome (E, K, T) is thus attained than in the case of a longer duration of the therapy. Therefore, for example, the following is true:

Outcome (E, K, T)=Result: (Costs×Time)=E:(K×T).

The recording of the time for which the treatment has already been carried out is easily possible in an automated fashion, for example by forming differences between the date of the calculation of the Outcome and the date of the first prescription or the start of the therapy or of the training program.

In a further embodiment of the present methods, the compliance C of the patient, i.e. his readiness and capacity to carry out the therapeutic measures as prescribed, are also taken into account in the measurement of the success of treatment. An example of the recording of a numerical measure of the compliance of a patient, in particular in a telemedical treatment process, is known from DE 101 36 759.7. Here too, the compliance can be expressed by means of a numerical measure, preferably in relative units, for example as a percentage of the services provided according to the prescription, and can be recorded in an automated fashion during the execution of the training program.

It is appropriate to take into account the compliance of the patient in a measurement of the Outcome of a therapy for the following reasons: on the one hand, a benchmark for the efficiency of a medical service provider is unfair if factors are included on which the service provider has no influence. Such a factor is the cooperativeness of the patient, which is expressed in the form of the numerical measure of compliance. A poor Outcome result must therefore to a certain extent be offset in favor of the service provider if there is insufficient compliance of the patient. This may be done, for example, by way of the following calculation rule:

Outcome (E, K, T, C)=Result: (Costs×Time×Compliance).

In this example of a calculation rule, if there is 100% compliance (C=1), the Outcome is the same as if the compliance is not taken into account. If the patient's compliance is poor, the Outcome will drop, but will be raised again computationally by the division by a number C<1.

On the other hand, it may in fact be part of the objective, and thus the responsibility of the medical service provider, to ensure or improve the patient's compliance by means of suitable measures. This is frequently the case in what are referred to as Disease Management Services for chronic illnesses such as, for example, diabetes or asthma. In such therapies, poor compliance of the patient must be included in the calculation as an attenuating factor for the Outcome, the numerical measure C of the compliance being present in the numerator of the calculation formula or being added to the Outcome:

Outcome˜E×C, or Outcome˜E+C.

A low compliance C<1 reduces the Outcome here. This form of calculation punishes the service provider for the fact that it has not succeeded in motivating the patient. It is implicitly assumed here that the result would have been better if there had been a higher level of compliance. If appropriate, the values for Outcome and Compliance must also be analyzed separately. Thus, for example given a very poor result, i.e. E near to zero, but good compliance, i.e. C near to 1, it is necessary to conclude that the treatment strategy is incorrect.

In the present methods, calculation rules which are suitable for different therapies are preferably already provided by connecting the first data processing station to a database with this information. The same applies to cost elements which are assigned to individual therapies, divided into costs which can be influenced and costs which cannot be influenced, and into associated key variables, if appropriate with the respective weighting factors. The user is provided here with a computer-based user interface with which he is supported in making his selection of the numerical measures and the definition or selection of the calculation rule for the calculation of the Outcome. The calculation rule and the associated categories are preferably selected here by means of customary computer input mechanisms, for example clicking on a mouse, drag and drop, etc. The calculation rule which is created or selected is automatically stored here in a memory and the access operations to the data which is necessary to calculate the Outcome are performed by the data processing station. Finally, such a computer module can also be used to carry out an automated configuration of the second data processing station for a home-based training workstation for the patient to carry out the rehabilitation measures with software-based and hardware-based methods/devices which provides the methods/devices for acquiring the numerical measures necessary for the Outcome calculation.

The present system for carrying out the methods is composed of a first data processing station for the user or therapist as well as at least one second data processing station at the location at which the therapeutic measures are carried out. The second data processing station is preferably located within the domestic environment of the patient so that he can carry out the therapeutic measures prescribed for him in his domestic environment. First and second data processing stations are networked to one another at least temporarily in order to exchange data. In the first data processing station, a first program module is provided for the interactive definition of the treatment objective and for providing a calculation rule for the success of treatment of the therapy with respect to the treatment objective. The first module has access to one or more databases in which corresponding calculation rules and numerical measures for various therapies are stored. Furthermore, the first module is constructed in such a way that it can set up a link to a second module in the second data processing station and can instruct the latter to record specific numerical measures. The second module is configured here in such a way that it carries out the recording of the numerical measures requested by the first module or coordinates them and transmits these numerical measures to the first module.

The present methods and the associated system permit automated and reliable measurement of the success of treatment of a medical therapy in standardized relative units, in particular in the rehabilitation or the disease management of chronic illnesses. The methods and the system can easily be embedded in the customary clinical workflow or the disease management service. For this purpose, use is made of a data networking structure with at least two data processing stations which also provide the data for the automated calculation in the course of the therapy in a spatially distributed treatment process, in particular in a telemedical therapeutic process or a telemedical rehabilitation measure. In the case of multiple measurements during the treatment, a quantified therapy profile check is also made possible by means of continuous monitoring of the Outcome profile. Given significant deviations of this Outcome profile from a predefinable reference curve, it is also possible to transmit a message to the user, i.e. the attending physician or therapist, in order to possibly discontinue or change the therapy. In one particularly advantageous embodiment, a cost-oriented measurement of the success of therapy takes place, which measurement is particularly informative given the increasing cost pressures in health services.

The methods include the provision of an infrastructure composed of databases and a computer workstation network for acquiring, within the scope of a spatially distributed therapeutic process, the data which is necessary to calculate the Outcome.

### BRIEF DESCRIPTION OF THE DRAWINGS

The present methods and the associated system are explained once more below with reference to exemplary embodiments in conjunction with the drawings without restricting the general inventive idea. In the drawings:

FIG. 1 shows an example of the infrastructure composed of databases and data processing stations with the method steps executed therein; and

FIG. 2 shows an example of possible ways of recording the numerical measures at the second data processing station which are required for a calculation rule.

### DETAILED DESCRIPTION OF THE PREFERRED EMBODIMENTS

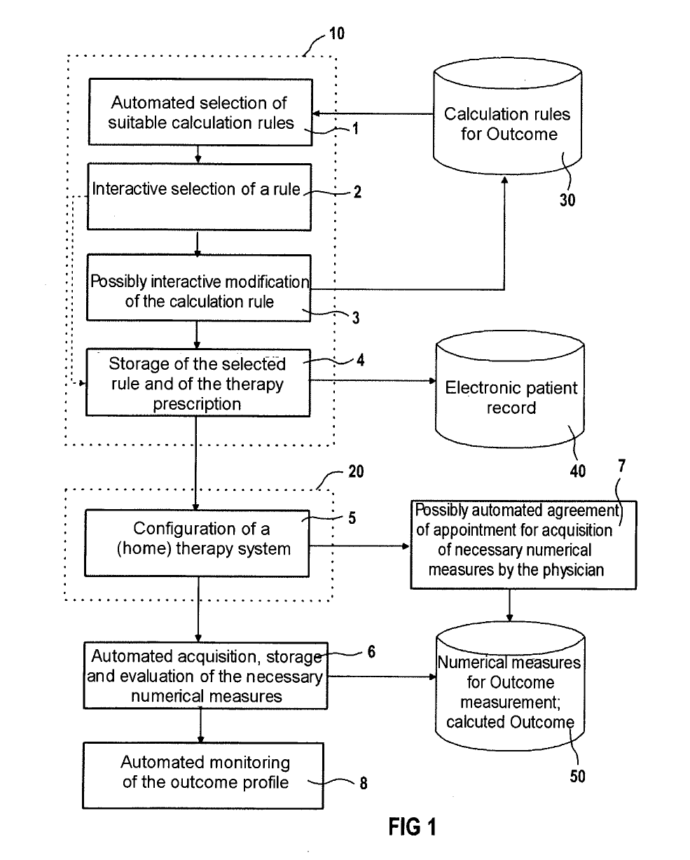

FIG. 1 shows, by way of example, individual method steps for the execution of the present methods according to a possible embodiment, and the associated data processing stations and databases. The method which is illustrated by way of example starts at a computer workstation (first data processing station 10) for therapy planning and therapy profile checking. This computer workstation 10 includes a software module which supports the user in composing, at the start of the treatment, a suitable Outcome calculation scheme for the therapeutic process which has been prescribed for the patient, assigning it to this therapeutic process and providing means for the automated recording and evaluation of the measured data necessary for this. For this purpose, one or more databases 30, which contain a list of the relevant cost blocks for each therapy, are stored on the computer workstation 10 in the present example. These costs are already preclassified into fixed costs and variable costs according to the preceding definition. Furthermore, for each cost block, the database 30 contains a numerical measure of the size of the costs, for example in currency units. These numerical measures can be stored as a presetting in the database, but can also be adapted individually to the conditions of the respective service provider or the respective institution. Furthermore, there is a database with key variables for the measurement of cost blocks and their assignment to the cost block. The database can also contain weighting factors which are assigned to each key variable and which permit the key variables to be converted into currency units.

In the text which follows, a simplified example will be adopted of a patient with cognitive problems after a stroke, who is to be treated by means of a telemedically administered training program which is carried out at his home on a computer training workstation (second data processing station 20). In this example, fixed cost elements, which can hardly be influenced, are the one-off provision of the computer training workstation 20 and the familiarization of the patient, the initial examination and classification of the patient in terms of his deficit, the first composition, prescription and transmission of a training program which is optimum for the individual. On the other hand, relevant variable cost elements for the calculation of a cost-oriented Outcome are the personal sessions with the patient, the evaluation of alarm messages and information messages from the training sessions, the number of routine assessments of the training results and the progress of training, the adaptation and represcription of the training program and the refamiliarization of the patient or outpatient treatments.

Suitable measurable or countable key variables S(i) for the variable cost elements of this rehabilitation process are therefore the duration of the treatment, the number of consultations with the physician/therapist, the number of days spent hospitalized in a rehabilitation clinic, the number of alarm messages or information messages, the number of manual training program modifications by the physician or therapist, the number of log-ins of the physician or therapist into a patient account and the number of access operations to the electronic patient files of the home therapy system as well as possibly the number of transmitted data items between the home and clinic or doctor's practice in the case of a data quantity-related billing mode of the service provider. For example, the time which the patient spends on carrying out the training alone, and thus the number of training units carried out, are not relevant to the health costs, and are thus unsuitable for the measurement. These key numbers S(i) can be recorded very easily in an automated fashion so that the inclusion of the costs in the method does not incur any greater expenditure.

In a user interface of the computer workstation 10, all the cost blocks which are relevant to this program are automatically represented for the user, i.e. the physician or therapist, after the selection of a therapy or of a training program, and the irrelevant cost blocks are deleted. As an option, the relative proportion which the cost blocks make up of the overall costs may be represented graphically for all the cost blocks, for example in the form of a bar chart. The user can then mark with the customary simple interactive operator control device, i.e. by clicking on a mouse, ticking a list, drag and drop, etc., those cost blocks which he wishes to include in the calculation of the Outcome. In addition, the user can mark which categories, in addition to the treatment result, i.e. costs, duration of treatment and compliance, are to be included in the calculation of the Outcome. With the selected predefined items as peripheral condition, a selection of possible calculation rules is then displayed to the user, the calculation rules being stored in the database 30 with respect to the particular therapies and categories. From this selection of the possible calculation rules, the user can in turn mark one which appears suitable for him.

The operator control can optionally also be carried out in a different order, a list of Outcome calculation rules which are relevant to the selected training program being automatically displayed to the user (Step 1), from which rules he selects one (Step 2). As a result, the user is then provided with an overview of all the numerical measures which will be acquired in future for the calculation of this Outcome with the selected calculation rule, and evaluated. As an option, it is possible to provide that the user indicates the selected calculation rule even further by selecting an assigned set of numerical measures.

In a further possible refinement of the method, the user can edit the calculation rules for the success of treatment, i.e. create new ones or change them, and add the newly created calculation rules to the database 30 of possible calculation schemes (Step 3).

After the user has decided, in Step 2, on a calculation rule of the Outcome, the rule is stored in an assignment to the patient and to the selected training program (Step 4). This data is stored in a database 40 or an electronic patient record (EPR). The computer 10 then automatically creates the access operations (references to files, etc.) to the numerical measures necessary for the calculation. The program module can also initiate, in an automated fashion, measures for providing all the means which are necessary to measure the numerical measures necessary in the course of the therapy. These may include, for example, the enabling of measurement modules provided for the purpose in the patient-end training software (second program module) at the second data processing station 20 (computer training workstation), such as, for example, quality of life questionnaires, computer-based capability tests, etc.

In the present method, the configuration of the computer training workstation which is to be installed at the patient is also preferably carried out automatically, for example by way of automatic specification of the sensor modules which are also to be supplied (Step 5), after definition of the training program and of the calculation rule. This also includes the creation of a timetable for the measurement of the key variables and the automatic triggering of the measurement operation for the measurement of the numerical measures of the success of treatment. A check to determine whether new numerical measures are present is preferably carried out here by the program module of the first data processing station 10 at regular time intervals or at predefined measuring times.

Alternatively, when new numerical measures arise, a notification to the program module can be transmitted or a new calculation of the Outcome can be triggered in a manually interactive fashion on the screen. The numerical measures for the calculation of the success of treatment are acquired automatically at the computer training workstation of the patient 20, stored and evaluated by the first data processing station in accordance with the selected calculation rule (Step 6). The numerical measures acquired in the course of the treatment and Outcome values are stored in a database 50 and are available at any time for representation, for example in a graphic form or in the form of a list, on the computer workstation 10. The Outcome profile over time is displayed automatically on the screen (Step 8). If appropriate, given significant deviations from a reference curve, an alarm can also be issued at one of the data processing stations 10, 20 involved.

If the Outcome calculation program requires numerical measures which cannot be acquired in an automated fashion in the course of the execution of the therapy, the program can automatically, at predefined times, arrange an appointment with the respective persons, for example physician and patient, at which the necessary numerical measures are acquired, for example by measuring a capability deficit in a rehabilitation clinic (Step 7).

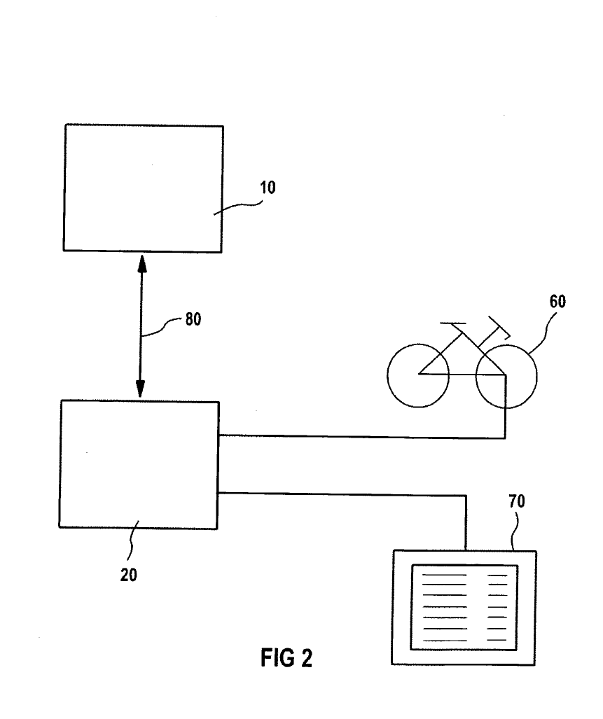

FIG. 2 shows an example of networking of the two data processing stations 10, 20 via a network 80, the data processing station 20 at the patient's home being also connected in this example via an interface to an ergometer 60 which can be used to acquire numerical measures of the patient's performance capabilities. Furthermore, this figure shows the acquisition of numerical measures by use of a questionnaire 70. This can be displayed on the screen of the computer training workstation at the necessary measuring time in order to prompt the patient to process it. This computer training workstation 20 also prescribes to the patient the training program which he is to run through within the scope of the therapy.

The present method and the associated system are used in particular in the treatment by way of innovative telemedical forms of treatment and in the care of the patient at home (Home care). A number of aspects of the present method are particularly adapted to such applications. The suitable configuration of a home-based training workstation for the acquisition of numerical measures which are necessary for the Outcome calculation is thus an absolutely necessary precondition to be able to measure Outcomes measurement in the context of telemedical rehabilitation with a logistically acceptable degree of expenditure.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A method for measuring the success of treatment of a medical therapy having the following steps:
defining at least one treatment objective of the therapy at a first data processing station;
provisioning a calculation rule for the success of treatment of the therapy with respect to the treatment objective, which indicates success of treatment as a function of recordable numerical measures, the provisioning taking place at the first data processing station by selection from calculation rules assigned to the therapy and automatically called from a database;
recording at least one first numerical measure describing an initial state of a person to be subjected to the therapy, with respect to the treatment objective;
storing data covering at least the treatment objective, the calculation rule and the first numerical measures;
recording at least one second numerical measure describing the success of treatment of the person at least one of during and at the end of the therapy, the recording occurring via a second data processing station;
calling the stored data and automatically calculating the success of treatment using at least one of the calculation rule at the first data processing station and second data processing station, wherein at least one of the second numerical measures and the success of treatment, possibly already calculated at the second data processing station, is transmitted to the first data processing station via a network; and
representing the success of treatment at the first data processing station.

2. The method for determining the success of treatment of a medical therapy as claimed in claim 1, wherein a calculation rule indicating the success of treatment as a function of at least one of the costs, the costs and the duration of the therapy and the compliance of the patient is provided at a computer workstation.

3. The method as claimed in claim 1, wherein the calculation rule is a comparison rule according to which recorded numerical measures are compared using a look-up table, stored in a database and linking the recorded numerical measures to a value of the success of treatment.

4. A method for measuring the success of treatment of a medical therapy comprising the following steps:
defining at least one treatment objective of the therapy at a first data processing station;
provisioning a calculation rule for the success of treatment of the therapy with respect to the treatment objective, which indicates the success of treatment as a function of recordable numerical measures, the provisioning taking place at the first data processing station, the provisioning of the calculation rule including the selection of categories to be included in the calculation, and the categories and calculation rules assigned to the selection being called from a database;
recording at least one first numerical measure describing an initial state of a person to be subjected to the therapy, with respect to the treatment objective;
storing data covering at least the treatment objective, the calculation rule and the first numerical measures;
recording at least one second numerical measure describing the success of treatment of the person at least one of during and at the end of the therapy via a second data processing station;
calling the stored data and automatically calculating the success of treatment using the calculation rule at at least one of the first data processing station and the second data processing station, wherein at least one of the second numerical measure and the success of treatment, having possibly been calculated at the second data processing station, is transmitted to the first data processing station via a network; and
representing the success of treatment at the first data processing station.

5. The method for determining the success of treatment of a medical therapy as claimed in claim 4, wherein a calculation rule indicating the success of treatment as a function of at least one of the costs, the costs and the duration of the therapy, and the compliance of the patient is provided at a computer workstation.

6. The method as claimed in claim 4, wherein the calculation rule is a comparison rule according to which recorded numerical measures are compared using a look-up table, stored in a database and linking the recorded numerical measures to a value of the success of treatment.

7. The method as claimed in claim 4, wherein the categories in the database are assigned to treatment objectives and only the categories relevant to the respective treatment objective are offered for selection.

8. The method as claimed in claim 4, wherein the selection of the calculation rule and of the associated categories is carried out by a customary computer input mechanism.

9. A method for measuring the success of treatment of a medical therapy having the following steps:
defining at least one treatment objective of the therapy at a first data processing station;
at least one of provisioning and inputting a calculation rule for the success of treatment of the therapy with respect to the treatment objective, the rule indicating the success of treatment as a function of recordable numerical measures and the at least one of provisioning and inputting occurring at the first data processing station, wherein the success of treatment is calculated with respect to the costs incurred for it, and wherein the costs are taken into account with key variables, easily recordable and proportional to the costs;
recording at least one first numerical measure describing an initial state of a person to be subjected to the therapy, with respect to the treatment objective;
storing data covering at least the treatment objective, the calculation rule and the first numerical measures;
recording at least one second numerical measure describing the success of treatment of the person at least one of during and at the end of the therapy using a second data processing station;
calling the stored data and automatically calculating the success of treatment using the calculation rule at at least one of the first data processing station and the second data processing station, wherein at least one of the second numerical measures and the success of treatment, having possibly been calculated at the second data processing station, is transmitted to the first data processing station via a network; and
representing the success of treatment at the first data processing station.

10. The method as claimed in claim 9, wherein, in the case of the costs, only the costs influenceable by at least one of the service provider and the patient are included in the calculation.

11. The method as claimed in claim 9, wherein cost proportions and key variables relating to the selected therapy are called from a database and displayed for selection in an automated fashion.

12. The method for determining the success of treatment of a medical therapy as claimed in claim 9, wherein a calculation rule indicating the success of treatment as a function of at least one of the costs, the costs and the duration of the therapy, and the compliance of the patient is provided at a computer workstation.

13. The method as claimed in claim 9, wherein the calculation rule is a comparison rule according to which recorded numerical measures are compared using a look-up table, stored in a database and linking the recorded numerical measures to a value of the success of treatment.

14. The method as claimed in claim 9, wherein the provision of the calculation rule includes the selection from calculation rules assigned to the therapy and automatically called from a database.

15. The method as claimed in claim 9, wherein the provision of the calculation rule includes the selection of categories included in the calculation, and wherein the categories and calculation rules assigned to the selection are called from a database.

16. The method as claimed in claim 15, wherein the categories in the database are assigned to treatment objectives and only the categories relevant to the respective treatment objective are offered for selection.

17. The method as claimed in claim 15, wherein the selection of the calculation rule and of the associated categories is carried out by customary computer input mechanisms.

18. The method as claimed in claim 1, wherein the calculation rule is provided in a form editable by the user and wherein a calculation rule, at least one of newly created and modified by the user, is added to the database.

19. The method as claimed in claim 1, wherein the second data processing station is configured for the automated recording and transmission of the second numerical measures describing the success of treatment to the first data processing station.

20. The method as claimed in claim 1, wherein the recording of the second numerical measures describing the success of treatment at least one of is brought about by the first data processing station and takes place at predefinable time intervals.

21. The method as claimed in claim 1, wherein at least one of the second numerical measures are recorded using a device connected to the second data processing station.

22. The method as claimed in claim 1, wherein, in order to record second numerical measures which cannot be acquired in an automated fashion, at least one of the first and second data processing station generates a notification for the agreement of an appointment for the acquisition of these second numerical measures.

23. The method as claimed in claim 1, wherein partial objectives, the attainment of which is monitored by multiple calculation of the success of treatment during the duration of the treatment, are predefined at the first data processing station.

24. The method as claimed in claim 23, wherein the successes of treatment, measured at various times, are stored and represented in the form of a profile curve.

25. The method as claimed in claim 24, wherein a reference curve is predefined for the profile and when the profile curve deviates from the reference curve by a predefinable value, an alarm is issued.

26. The method as claimed in claim 1, wherein the success of treatment is calculated with respect to the time taken up by the treatment.

27. The method as claimed in claim 1, wherein the success of treatment is calculated taking into account the compliance of the patient.

28. A system for carrying out a method for measuring the success of treatment of a medical therapy, comprising:
a first data processing station having a first module for defining a treatment objective, for providing a calculation rule and for automated execution of a calculation with reference to at least one of transmitted and stored numerical measures, wherein the first module is connected, for provisioning of the calculation rule, to a database containing calculation rules assigned to different therapies and treatment objectives; and
a second data processing station, connected to the first data processing station at least temporarily via a network and including a second module for the automatic recording and transmission of numerical measures to the first module of the first data processing station at predefinable times.

29. The system as claimed in claim 28, wherein the second data processing station is connected, via at least one interface, to a device for measuring the numerical measures.

30. The method as claimed in claim 2, wherein the calculation rule is a comparison rule according to which recorded numerical measures are compared using a look-up table, stored in a database and linking the recorded numerical measures to a value of the success of treatment.

31. The method as claimed in claim 4, wherein the calculation rule is a comparison rule according to which recorded numerical measures are compared using a look-up table, stored in a database and linking the recorded numerical measures to a value of the success of treatment.

32. The method as claimed in claim 4, wherein the selection of the calculation rule and of the associated categories is carried out by at least one of clicking on a mouse and by use of drag and drop.

33. The method as claimed in claim 9, wherein cost proportions and key variables relating to the selected therapy are called from a database and displayed for selection in an automated fashion.

34. The method as claimed in claim 15, wherein the selection of the calculation rule and of the associated categories is carried out by at least one of clicking on a mouse and by use of drag and drop.

35. The method as claimed in claim 4, wherein the calculation rule is provided in a form editable by the user and wherein a calculation rule, at least one of newly created and modified by the user, is added to the database.

36. The method as claimed in claim 4, wherein the second data processing station is configured for the automated recording and transmission of the second numerical measures describing the success of treatment to the first data processing station.

37. The method as claimed in claim 4, wherein the recording of the second numerical measures describing the success of treatment at least one of is brought about by the first data processing station and takes place at predefinable time intervals.

38. The method as claimed in claim 9, wherein the calculation rule is provided in a form editable by the user and wherein a calculation rule, at least one of newly created and modified by the user, is added to the database.

39. The method as claimed in claim 9, wherein the second data processing station is configured for the automated recording and transmission of the second numerical measures describing the success of treatment to the first data processing station.

40. The method as claimed in claim 9, wherein the recording of the second numerical measures describing the success of treatment at least one of is brought about by the first data processing station and takes place at predefinable time intervals.

41. The method as claimed in claim 4, wherein at least one of the second numerical measures are recorded using a device connected to the second data processing station.

42. The method as claimed in claim 9, wherein at least one of the second numerical measures are recorded using a device connected to the second data processing station.

43. The method as claimed in claim 1, wherein, in order to record second numerical measures which cannot be acquired in an automated fashion, at least one of the first and second data processing station generates a proposal, for the agreement of an appointment for the acquisition of these second numerical measures.

44. A system for carrying out the method of claim 1, comprising:
a first data processing station having a first module for defining the treatment objective, for providing the calculation rule and for automated execution of the calculation with reference to at least one of transmitted and stored numerical measures, wherein the first module is connected, for provisioning of the calculation rule, to a database containing calculation rules assigned to different therapies and treatment objectives; and
a second data processing station, connected to the first data processing station at least temporarily via a network and including a second module for the automatic recording and transmission of numerical measures to the first module of the first data processing station at predefinable times.

45. A system for carrying out the method of claim 4, comprising:
a first data processing station having a first module for defining the treatment objective, for providing the calculation rule and for automated execution of the calculation with reference to at least one of transmitted and stored numerical measures, wherein the first module is connected, for provisioning of the calculation rule, to a database containing calculation rules assigned to different therapies and treatment objectives; and
a second data processing station, connected to the first data processing station at least temporarily via a network and including a second module for the automatic recording and transmission of numerical measures to the first module of the first data processing station at predefinable times.

46. A system for carrying out the method of claim 9, comprising:
a first data processing station having a first module for defining the treatment objective, for providing the calculation rule and for automated execution of the calculation with reference to at least one of transmitted and stored numerical measures, wherein the first module is connected, for provisioning of the calculation rule, to a database containing calculation rules assigned to different therapies and treatment objectives; and
a second data processing station, connected to the first data processing station at least temporarily via a network and including a second module for the automatic recording and transmission of numerical measures to the first module of the first data processing station at predefinable times.

47. The system as claimed in claim 44, wherein the second data processing station is connected, via at least one interface, to a device for measuring the numerical measures.

48. The system as claimed in claim 45, wherein the second data processing station is connected, via at least one interface, to a device for measuring the numerical measures.

49. The system as claimed in claim 46, wherein the second data processing station is connected, via at least one interface, to a device for measuring the numerical measures.
